(19) United States
(12) Patent Application Publication
(10) Pub. No.: US 2005/0125043 A1
(43) Pub. Date: Jun. 9, 2005

(54) Device for treating patients by means of brain stimulation

(76) Inventor: Peter Tass, Dusseldorf (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/507,959

(22) PCT Filed: Feb. 19, 2003
(86) PCT No.: PCT/DE03/00497

(30) Foreign Application Priority Data
Mar. 14, 2002 (DE) 10211766.7

(51) Int. Cl. A61N 1/18

(52) U.S. Cl. 607/45

(57) ABSTRACT

The invention relates to a device for treating patients by means of brain stimulation, an electronic component, and the use of said device and electronic component in medicine. Brain electrodes known in prior art, which are used for therapeutic permanent stimulation, have the disadvantage that as a non-physiological input in the area of the brain, for example the thalamus or the basal ganglions, permanent high-frequency stimulation can result in the affected neuron groups adapting thereto within a few years. Stimulation consequently has to be performed at a higher amplitude in order to obtain the same stimulating effect. Said disadvantages are eliminated by the inventive device comprising the following components: at least one brain stimulating electrode (2); at least one sensor (3, 2) measuring an electrical signal; control means (4) which detect the presence of a pathological signal and feed at least one pulse to the electrode (2) when the pathological signal occurs while turning off the pulse when the pathological feature discontinues.

The invention relates to a device for the treatment of patients by means of brain stimulation according to the preamble of claim 1, to an electronic component as well as to the use of the device and the electronic component in the practice of medicine.

In patients with neurological or psychiatric pathologies like for example Morbus Parkinson, essential tremors, dystonia or obsessive disorders, nerve cell groups in circumscribed regions of the brain, for example the thalamus and the basal ganglion, are pathologically active, for example excessively synchronous. In these cases a large number of neurons synchronously generate action potentials. That means that the associated neurons fire largely synchronously. With healthy patients the neurons in these regions of the brain fire qualitatively differently, for example, in an uncorrelated manner.

In Morbus Parkinson, the pathologically synchronous activity changes the neural activity in areas of the cerebral cortex, like for example in the primary motor cortex in that the rhythm of the latter can be forced so that as a consequence, muscular activity controlled by these regions can develop a pathological response, for example, a rhythmic trembling.

In patients who can no longer be treated by medicaments, the deep electrode implantation is indicated, depending upon whether the pathology is one side or is two-sided. A cable runs from the head to a so-called generator implanted under the skin and which includes a control device with a battery and which is generally implanted beneath the skin in the region of the collar bone. Through the use of the deep electrodes, a permanent excitation with a high frequency periodic sequence (with a frequency in excess of 100 Hz) of individual excitations, for example with rectangular pulses (pulse train) can be carried out. The purpose of this method is to suppress the firing of the neurons in the target region. This standard deep stimulation results in a reversible lesioning, that is a reversible alteration of the tissue. The effective mechanism, that is how the standard excitation functions, has still not been clarified sufficiently.

The previously used methods have, however, several drawbacks. For example, energy consumption for permanent stimulation is very high so that the generator including the battery must be replaced already after one to three years by an operation.

It is especially disadvantageous, however, that the high frequency continuous stimulation is a nonphysiological and therefore unnatural input in the region of the brain, for example the thalamus or basal ganglions which can give rise over the passage of several years to adaptation of the impacted neuron groups. To obtain the same stimulation effect, therefore, it is necessary to compensate with higher excitation amplitudes. The greater the amplitude of excitation the greater is the probability that the irritation will have a side effect upon neighboring areas, like dysarthria (articulation disorders), dysesthesia (in part very painful sensitivity or sensory phenomenon), cerebellar ataxie (inability to stand without assistance) or schizophrenic like symptoms, etc. These side effects can be intolerable by patients. The treatment then loses in these cases its effectiveness after a few years.

It is therefore the object of the invention to provide a device which enables a treatment in which the symptoms of the respective disorder can be reduced or completely eliminated. However, the activity should not negatively impact upon the nerve cell group nor suppress the activity thereof but should bring a healthy functional state closer. Furthermore, the side effects like for example those already mentioned such as dysarthria, dysesthesia, cerebellar ataxie or schizophrenic like symptoms which result from the methods of the prior art, should be eliminated or at the very least reduced.

Starting from the preamble of claim 1, the object is obtained according to the invention with the features of the characterizing part of claim 1.

With the device according to the invention it is now possible to treat patients without resulting in an adaptation to unphysiological continuous irritation like the above mentioned side effects which are thereby reduced or suppressed. Through the use of the device according to the invention, in addition, the battery consumption or current consumption can be drastically reduced, such that the battery need be replaced less often or need be charged less frequently.

Advantageous features of the invention are given in the dependent claims.

The drawing shows an exemplary embodiment of the device according to the invention.

It shows:

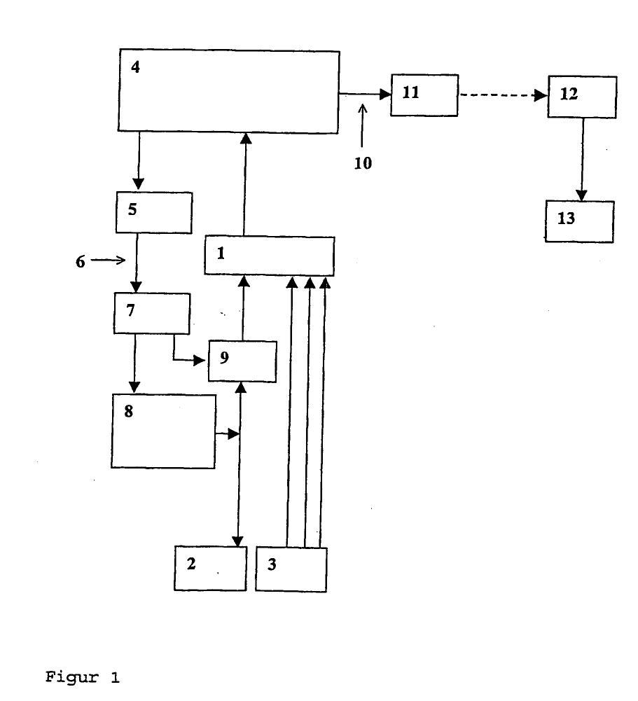

FIG. 1: a block diagram of the device.

The device according to the invention illustrated in FIG. 1 comprises an isolating amplifier (1) to which at least one electrode (2) and sensors (3) for detecting physiological measurement signals are connected. The isolating amplifier is in turn connected with a unit (4) for signal processing and control and which is connected to an optical transmitter (5) for the stimulation. The optical transmitter (5) is connected by lightwave guide (6) with an optical receiver (7) which is in connection with a simulator unit (8) for signal generation. The simulator unit (8) for signal generation is in connection with the electrode (2). At the input region to the electrode (2) in the isolating amplifier (1) there is a relay (9) for a transistor. The unit (4) is connected by a conductor (10) with a telemetric transmitter (11) which is connected with a telemetric receiver (12) located externally of the implanted device and connected in turn with a means (13) for visualizing, processing and storing the data.

As sensors (3), for example, epicordical electrodes, deep electrodes, brain electrodes or peripheral electrodes can be used.

The electrode (2) can have at least two wires at whose ends a potential difference can be applied for the purposes of stimulation. It can thus be a macro electrode or a micro electrode. Additionally but not compulsorily, a potential difference can be measured by the electrode (2) to detect a pathological activity. In a further embodiment, the electrode (2) can also be comprised of more than two individual wires which can permit the detection of a measurement signal in the brain as well as apply the stimulation. For example, four wires can be provided in a conductor cable, whereby between different ends of the wires, a potential difference can be applied or measured. In this manner, the size of the derived or stimulated target region can be varied. The number of wires from which the electrode is formed is limited as to its upper value only by the associated thickness of the cable which is to be introduced into the brain so that the least possible amount of brain material will be damaged. Commercial electrodes encompass four wires, although they can have also five, six or more wires although only three wires can be used as well.

For the case in which the electrode (2) encompasses more than two wires, at least two of these wires can also function as the sensor (3) so that in this special case an embodiment is provided in which the electrode (2) and the sensor (3) are combined in a single component. The wires of the electrode (2) can have different lengths so that they penetrate to different depths in the brain. If the electrode (2) is comprised of n wires, a stimulation can be effected via at least one pair of wires, whereby the pair formation can involve different subcombinations of wires. Aside from this component, sensors (3) which are not included in the component with the electrode (2) can be provided.

A unit for signal processing and control (4) can encompass means for a univariate and bivariate data processing like for example that in “Detection of n:m phase locking from noisy data: Application to Magnetoencephalography” of P. Tass et al in Physical Review Letters, 81,3291 (1998).

According to the invention the device is equipped with means which can recognize the signals of the electrode (2) and/or the sensors (3) as pathological and in the case of the presence of a pathological pattern can output through the electrode (2) excitation signals which effect a brief suppression of the pathological neuronal activity or so modify the pathological neuronal activity that it approaches more closely the natural physiological activity. The pathological activity differs from the healthy activity by a characteristic variation in its pattern and/or its amplitude.

The means for recognizing the pathological pattern is thus a computer which processes the measured signals from the electrode (2) and/or the sensor (3) and compares them with data stored in the computer. The computer has a data carrier which stores data which can be developed through a standardization or calibration procedure. For example this data can be detected through a series of test excitations which systematically vary the stimulation parameters and record and process the results of the stimulation as detected by the electrode (2) and/or the sensor (3) by means of the control unit (4). The detected results can be subjected to a univariate, bivariate and multivariate analysis by characterization of the frequency characteristics and the interaction, for example coherence, phase synchronization, directionality and excitation response characteristics as for example disclosed in P. A. Tass: “Phase resetting in medicine and biology. Stochastic Modelling and Data Analysis”, Springer Verlag, Berlin, 1999.

The device according to the invention encompasses, consequently, a computer which contains a data carrier which carries data with respect to the pathology picture which is comparable with the measurement data and in the case of arising pathological activity will output an excitation signal at the electrodes (2) so that a stimulation of the brain tissue results. The data stored in the data carrier of the pathological picture can be either person specific, obtained by standardization with the particular individual using optimal stimulation parameters, or a data pattern which is obtained from a collection of patients and represent typically arising optimal stimulation parameters. The computer recognizes the pathological pattern and/or the pathological amplitude.

The types of stimulation used for the treatment of the pathological findings are known to the artisan. They can for example be those described under 1. and 2., below, such as long periodic sequences of individual excitations or complex excitation sequences. Examples of these complex stimuli are on the one hand a double pulse which is comprised of two qualitatively different pulses, for example a strong pulse and a weak pulse and on the other hand a high frequency (greater than 100 Hz) or low frequency (between 5 and 20 Hz) consequent upon an individual pulse. As a consequence of the excitation used, the pathological activity in the case of the use of longer periodic sequences of individual excitations typically suppress the pathological activity while in the case of complex excitation sequences typically bring the activity closer to the natural nonpathologically activity or cause the activity to completely resume the normal nonpathological activity. The device according to the invention is so configured that in the case in which the electrode (2) and/or sensor (3) detects following the excitation an elimination of the pathological activity, the stimulation will be interrupted. For this purpose, the computer determines whether the pathological increase in amplitude or the pathologically increased resemblance to a particular pattern is present. The consequence is an analysis by the electronic circuitry of the data. As soon as the pathological features are again detected, the next stimulation is commenced in the same way. The switching on and switching off of the stimulation is effected either by a control unit or by two control units communicating with one another which are collected in the control unit (4) illustrated in FIG. 1.

The control unit (4) can be embodied with a chip or another electronic device with comparable computing power.

The control unit (4) controls the electrode (2) preferably in the following manner. The control data are delivered by the control unit (4) to an optical transmitter (5) for the stimulation and which controls the optical receiver (7) through the lightguide (6). Because of the optical coupling of the control signal applied to the optical receiver (7) there is a galvanic decoupling of the stimulation control from the electrode (2). This means that the pickup of noise signals by the electrode (2) from the signal processing and control unit (4) is prevented. As an optical receiver (7) a photocell can for example be considered. The optical receiver (7) produces signals which trigger the stimulator unit (8) and originate at the optical stimulation transmitter (5). Through the stimulator unit (8) the targeted stimuli are reproduced in the target region in the brain via the electrode (2). For the case in which the electrode (2) also provides a measurement of the stimulation, starting from the optical stimulation transmitter (5) through the optical receiver (7) the relay (9) is controlled which prevents the pickup of noise or stray signals. The relay (9) or the transistor ensues that the neuronal activity can be directly measured immediately following each stimulus without the overmodulation or overloading of the isolating amplifier. The galvanic decoupling need not always be effected by an optical coupling of the control signals and indeed other alternative control or couplings can be used. This can include for example an acoustic coupling for example in the ultrasonic range. A noise free control can also be realized for example with the aid of appropriate analog or digital filters.

In addition, the device of the invention can be connected preferably with means for visually displaying and processing the signals and for data storage (13) through the telemetric receiver (12). The unit (13) can then be capable of the univariate, bivariate or multivariate data analysis as has been described previously.

Furthermore, the device according to the invention can be connected through the telemetric receiver (13) [sic.] with an additional reference databank in order to accelerate for example the standardization process.

The invention is described in greater detail in the following.

According to the invention, the pathological neuronal activity (A) is measure through an electrode (2) like a (a) brain electrode, for example, a deep electrode, a (b) epicordical electrode or through (c) a muscular electrode and serves as a feedback signal and thus has a control signal for a need-controlled stimulation (B). The feedback is supplied through a conductor from the sensor (3) to the isolating amplifier (1). Alternatively the feedback signal can be transmitted—without the use of an isolating transformer—telemetrically. In the case of telemetric transmission, the sensor (3) is connected with the amplifier by a cable. The amplifier is connected with a telemetric transmitter by a cable. In this case, sensor (3) and the amplifier and telemetric transmitter can be implanted for example in the region of an extremity as to which there is concern, while the telemetric receiver is connected by a cable with the control unit (4). This means that, different from a standard permanent excitation, the activity is measured and the measurement signal is used as a trigger for a need-control stimulation.

For the measurement (A) of the neuronal activity the following different possibilities apply:

I. Measurement by the brain electrode (a) (Electrode (2), while in this case assumes the function of a sensor (3)), over which the stimulation is also effected. When electrode (2) is comprised of more than three wires, at least two of these wires can function as the sensor (3) whereby in this case, the stimulation is not effected through these wires.

II. Measurement of the neuronal activity of deep regions of the brain, like the thalamus or the basilgangleon through the deep electrode (a′) (sensor (3)), by means of which the stimulation is not effected. In this case, apart from the electrode (2) functioning as the deep electrode (a), a further deep electrode (a′) is used as the sensor (3).

III. Measurement of the neuronal activity which arises from the cortex of the brain, either through an implanted electrode (b) or preferably through a nontramatic epicordical electrode (b) (sensor (3)), that is an electrode which lies upon the brain and is fixed but does not penetrate into the tissue and in this manner derives a local electroencephalogram from a certain area of the brain cortex, for example, the primary motor cortex.

IV. With patients who suffer primarily from a tremor, a measurement of muscular activity can also be effected through electrode (c) (sensor (3), preferably telemetrically connected with the control unit (4)) in the region of the affected musculature.

The pathological neuronal activity can basically also arise in different neuron populations. For that reason, also a plurality of measured signals can be used through electrode (2) and/or sensor (3) to control the stimulation. Whenever in at least one of the neuron population a pathological feature of the activity is detected, an excitation is effected or triggered. The electrode (2) can also assume the function of a sensor (3). This enables a derivation of the activity of the neuron population at the treatment point of the electrode (2).

The measured signal or the measured signals serve as feedback signals. This means that a stimulation is effected as a function of the activity determined by the measured signal. Whenever a pathological feature of the neuron activity (that means pathologically increased amplitude or pathologically increased impressed activity pattern) commences and increases, the stimulation is effected.

The stimulation (B) can also be effected in various ways:

1. Need-determined stimulation with a high frequency pulse train (a pulse train greater than 100 Hz):

Whenever the pathological activity commences, a sufficiently long high frequency pulse train is applied. The sufficient length of the high frequency pulse train is determined by a standardization or calibration procedure. During the period which the relevant group of neurons requires to again develop the pathological activity, no stimulation is effected. In this manner the stimulation time is significantly reduced since even with heavily affected patients for periods of minutes and significantly longer for example there may not be pathological activity.

2. Need-controlled stimulation or the desynchronization of synchronized oscillation activity:

This process is employed when pathologically synchronized nerve cell activity develops in the target area (as determined through electrode (2)) (for example in Parkinsonism in the region of the thalamus) or in another area or muscle relevant to the pathology (determined by sensors (3)). This is determined for example in that measured signals from the electrode (2) and/or sensor (3) are filtered using a band pass filtering in the frequency range characteristic of the pathological activity. As soon as a band pass filter measurement signal exceeds a threshold value determined in the frame work of a standardization or calibration procedure, the next control pulse is transmitted via the control unit (4) from the optical transmitter (5) and is supplied through the lightwave guide (6) and the optical receiver (7) to trigger the electrode (2) to produce the excitation. The goal here is not, as with standard continuous stimulation, to suppress simply the firing of the neurons. Rather it is intended as a response to need, only to eliminate the pathologically increased synchronization of the nerve cell. That means that the nerve cell group in the target area is desynchronized although they are trained to remain active with respect to the production of action potentials. The relevant nerve cell thus are caused to fire more closely to their physiological and thus uncorrelated state instead of having their activity completely suppressed in a simple manner. For this purpose a variety of different desynchronizing processes which can be described collectively as the principle of “stochastic phase resetting”, can be used. This utilizes the fact that a synchronized neuron population, by the application of an electrical excitation of the correct intensity and duration can be desynchronized and the excitation can interrupt the pathological rhythmic activity in a vulnerable phase layer. These optimal stimulation parameters (intensity, duration and vulnerable phase) are determined in the frame work of a standardization procedure for example by systematic variance of these parameters and characterization of the stimulation sequence (e.g. the damping of the amplitude of the bandpass filter feedback signal). In the case of the use of the telemetric device 11-13, the standardization or calibration can be carried out through the use of so called phase resetting curves in an accelerated manner. The individual pulse stimulation is only efficient when the excitation in the vulnerable phase or near enough to the vulnerable phase that the stimulated activity is applicable. Alternatively, complex stimulation shapes can also be used. These presume a resetting stimulus (that is a stimulus which controls the dynamics of the neuron population to be stimulated, for example starting anew) and a desynchronizing pulse together. The advantage of this complex method is that the complex stimulation shapes can be called up independently from the dynamic state of the neuron population to be stimulated for desynchronization.

In the case of the use of individual excitations, the control unit (4), upon overstepping of the threshold value determined by the standardization by means of the electronic circuitry of the control unit (4) calculate the point in time that the vulnerable phase may arise based upon standard predictional algorithms so that the vulnerable phase will be met with sufficient precision. In the case of the use of complex excitation, the control unit (4) upon overstepping the threshold value determined by the standardization must only call up a new complex excitation of the same kind.

Simple excitations are for example

(a) Individual pulse stimulations.

Complex excitations are for example

(b) Double pulse stimulations,

(c) Stimulation with a resetting high frequency pulse train (greater than 100 Hz pulse train), following a desynchronizing individual pulse,

(d) Stimulation with a resetting low frequency pulse train—in the region of the pathological frequency for example in the case of Morbus Parkinson of about 5 Hz—, following a desynchronizing single pulse.

In a preferred embodiment the device is equipped with means for the wireless transmission of data like for example the measurement signal and stimulation control signal for data transmission from the patient to an external receiver for example for the purpose of monitoring therapy and optimizing therapy. In this manner it can be determined at an early stage whether the stimulation parameters used are no longer optimal. In addition, by a wireless transmission of data a reference databank can be accessed and at an early stage there can be a reaction to typical variance in the ability to effect excitation in the target tissue.

According to the invention, an electronic component is provided which can measure the occurrence and decay of a pathological feature of the electrical signal by the sensor (3, 2) and upon the development of the pathological feature can produce a pulse at the electrode (2) which can shut down when the pathological feature falls off. It encompasses in a preferred embodiment a univariate data processing and in addition a multivariate and/or bivariate data processing.

Preferably the electronic component is so configured that at least one of the univariate, bivariate and multivariate data processing is carried out by the method of statistical physics, the field of stochastic phase resetting being derived from the method of statistical physics.

The device according to the invention and the electronic component according to the invention can be used in the practice of medicine, preferably in the field of neurology and the field of psychiatry.

1. A device for the treatment of patients, comprising means for stimulating brain region,
characterized in that it encompasses the following components:
at least one electrode (2) for the stimulation of a brain region,
at least one sensor (3, 2) for measuring an electrical signal,
control means (4) which can recognize the development and decay of a pathological feature of the electrical signal which is measured by the sensor (3, 2). and upon the occurrence of the pathological feature can output at least one pulse to the electrode (2), the pulse being shut off upon the drop off of the pathological feature.

2. The device according to claim 1 characterized in that the control means (4) encompasses univariate data processing.

3. (currently amended) The device according to claim 1 characterized in that the control means encompasses a multivariate and/or bivariate data processing.

4. The device according to claim 2 characterized in that at least one of the univariate, bivariate and multivariate data processing operates with methods of statistical physics.

5. The device according to claim 4 characterized in that the method of statistical physics is a method from which the field of stochastic phase resetting derives.

6. The device according to claim 1 characterized in that the electrode (2) comprises at least two wires.

7. The device according to claim 6 characterized in that the electrode (2) functions as a lead away electrode.

8. The device according to claim 1 characterized in that the sensor (3) is an epicordical electrode, a deep electrode, a brain electrode, a muscular electrode, the electrode (2) or at least a component from this group.

9. The device according to claim 1 characterized in that the sensor (3) is connected with the control unit (4) through an isolating amplifier (1).

10. The device according to claim 1 characterized in that the electrode (2) is connected with the control unit (4) through an isolating amplifier (1).

11. The device according to claim 9 characterized in that it includes means for preventing an overmodulation of the isolating amplifier.

12. The device according to claim 11 characterized in that the means for preventing the overmodulation of the isolating amplifier (1) is a relay, a transistor or an electronic filter (9).

13. The device according to claim 1 characterized in that the control unit (4) is telemetrically connected with the sensor (3).

14. The device according to claim 1 characterized in that the stimuli to the electrode (2) are supplied through a galvanically decoupled coupling (5).

15. The device according to claim 14 characterized in that the means for the galvanically decoupled coupling (5) of the stimuli include an optical transmitter and an optical receiver which transmits signals to the electrode (2).

16. The device according to claim 1 characterized in that the control unit (4) is connected with a telemetric transmitter (11).

17. The device according to claim 16 characterized in that the telemetric transmitter (11) is connected with a telemetric receiver (12).

18. The device according to claim 17 characterized in that the telemetric receiver (12) is connected to means for visually displaying, processing and storing the data (13).

19. The device according to claim 18 characterized in that the means for processing the data encompasses a univariate data processing.

20. The device according to claim 18 characterized in that the means for processing the data encompasses a multivariate and/or bivariate data processing.

21. The device according to claim 1 characterized in that the means for processing the data includes at least one of a univariate, bivariate and multivariate process for data processing with the methods of statistical physics.

22. The device according to claim 21 characterized in that the method of statistical physics is that from which the field of stochastic phase resetting derives.

23. The device according to claim 1 characterized in that the electrode (2) and the sensor (3) are at least partly encompassed in a housing.

24. An electronic component characterized in that it can recognize the occurrence and fall off of a pathological feature of an electrical signal which is measured by a sensor (3, 2) and by the occurrence of the pathological feature outputs at least one pulse to the electrode (2) which pulse is shut off upon fall off of the pathological feature.

25. The electronic component according to claim 24 characterized in that it encompasses a univariate data processing.

26. The electronic device according to claim 24 characterized in that it encompasses a multivariate and/or bivariate data processing.

27. The electronic component according to claim 25 characterized in that it operates with at least one of the univariate, bivariate and multivariate processes of data processing of the method of statistical physics.

28. The electronic component according to claim 27 characterized in that the method of statistical physics is that from which the field of stochastic phase resetting derives.

29. The use of the device according claim 1 in the practice of medicine.

30. The use of the electronic component according to claim 24 in the practice of medicine.
